(19) United States
(12) Patent Application Publication
(10) Pub. No.: US 2011/0060174 A1
(43) Pub. Date: Mar. 10, 2011

(54) CATALYST, A PROCESS FOR SELECTIVE HYDROGENATION OF ACETYLENE TO ETHYLENE AND A METHOD FOR THE MANUFACTURE OF THE CATALYST

(75) Inventors: Felix Studt, San Francisco, CA (US); Jens Kehlet Nørskov, Los Altos, CA (US); Claus Hviid Christensen, Lynge (DK); Rasmus Zink Sørensen, Copenhagen O (DK); Frank Abild-Pedersen, Menlo Park, CA (US); Thomas Bligaard, Kvistgard (DK)

(73) Assignee: DANMARKS TEKNISKE UNIVERSITET - DTU, Kongens Lyngby (DK)

(21) Appl. No.: 12/811,801

(22) PCT Filed: Jan. 7, 2009
(86) PCT No.: PCT/DK09/50009

§ 371 Date: Nov. 19, 2010

(30) Foreign Application Priority Data
Jan. 7, 2008 (DK) PA 2008 00020

(51) Int. Cl. B01J 23/825; C07C 5/09

(52) U.S. Cl. 585/260; 502/335; 502/259; 502/174; 502/328

(57) ABSTRACT

Provided is a catalyst including a mixture of metal A selected from Fe, Co and Ni, and metal B selected from Zn and Ga, and a support material, where the two metals are present in an intermetallic composition; A method for the manufacture of the catalyst; and the use of the catalyst for the selective hydrogenation of acetylene to ethylene in a gas mixture including acetylenic impurities and hydrogen, and one or more of ethylene and carbon monoxide. The catalyst has a high selectivity and is based an easily available metal compounds.

### TECHNICAL FIELD

The invention relates to novel catalysts and to methods of making supported intermetallic compositions exhibiting improved hydrogenation catalyst performance. According to a further aspect, the invention relates to processes for hydrogenation, generally, and, particularly, selectively hydrogenating acetylene to ethylene employing supported intermetallic catalysts prepared by the preparation method of this invention.

### BACKGROUND

Acetylenic impurities are byproducts in ethylene processes and they act as poisons to the catalyst for further processing. The poisonous components are only tolerable in amounts of about 5 ppm and effective catalysts for removing these components are needed. The most frequently used method for the removal processes are selective hydrogenation.

Preferably, the content of the acetylenic impurities in the feed for further processing should be less then 5 ppm (more preferably less than 1 ppm). However the initial concentration of the components, as received from the ethylene recovery processes may often exceed 1 wt. %. Therefore, further reduction of the concentration is required. The typical method for reducing the concentration is selective catalytic hydrogenation of the byproducts.

U.S. Pat. No. 7,247,760 (Cheung et al.) discloses a process for selective hydrogenation of acetylene to ethylene in the presence of a catalyst containing Pd and Ag supported on Al₂O₃. The catalyst is prepared by contacting a composition comprising palladium, silver and a support material (preferably alumina) with a liquid composition comprising an iodide component such as ammonium iodide followed by calcinating at 1000° F. (538° C.). Alloys of Pd (about 10,000 USD/kg in 1998) and Ag (about 150 USD/kg) are often too expensive to be commercially attractive.

EP 1 834 939 (Giedigkeit et al.) discloses a catalyst containing Pd and Ga and the use of such catalyst for the for selective hydrogenation of acetylene to ethylene. Specifically mentioned catalysts are PdGa and Pd₃Ga₇. The disadvantage of Pd-containing catalysts is the price of Pd.

U.S. Pat. No. 5,364,998 (Sarrazin et al.) discloses a process for the selective hydrogenation of unsaturated hydrocarbons using a catalyst containing a group VIII metal—including Ni, Pd, Pt, Rh and Ru—and an element chosen from within the group constituted by gallium and indium. Due to the costs such catalysts are less interesting for commercial use.

Rodriguez et al., Journal of Catalysis 171, 268-278 (1997), discloses Zn containing Ni-based catalysts for selective hydrogenation of acetylene. According to Rodriguez, Zn is included in the supporting carrier and is not included in an intermetallic composition with Ni.

U.S. Pat. No. 4,507,401 (Dubois et al.) discloses a catalytic composition comprising a Group VIII metal, such as Ni, and a metal chosen from the group comprising B, Al, Ga, In, Si, Ge, Sn or combinations of these metals. However, the only exemplified catalyst compositions of U.S. Pat. No. 4,507,401 contain Ni as one metal and either Si, Ge, B or Al as the other metal or Rh as one metal and Ge as the other metal. U.S. Pat. No. 4,507,401 does not disclose that the catalyst can be useful for the selective hydrogenation of acetylene to ethylene or any evidence that catalyst compositions comprising Fe, Co or Ni as one metal and either Zn or Ga as the other metal may be used as a catalyst.

Alloying an active metal with a second active or inactive metal can change the catalytic performance drastically. For example, with a mono-metallic palladium catalyst it has been demonstrated that the selectivity was substantially improved by adding silver to the palladium phase.

Considering the very high price of palladium such improvement using silver as proposed in U.S. Pat. No. 7,247,760 or gallium as proposed in EP 1 834 939 is a useful approach to a achieve cheaper and more efficient catalyst. However, as the price of palladium is very high and the price of silver is also rather high, there is still a need for a commercially attractive and efficient selective catalyst for the hydrogenation of acetylene to ethylene in commercial ethylene materials.

### DISCLOSURE OF THE INVENTION

It has now, surprisingly, been found that substantially cheaper and still efficient selective hydrogenation catalysts of the above mentioned type with two metals on a support material (C) can be obtained when the two metals are selected from a group A and a group B wherein the group A metal is Fe, Ni or Co and the group B metal is Zn or Ga.

Accordingly the present invention relates to a catalyst comprising a mixture of metal A selected from the group consisting of Fe, Co and Ni and metal B selected from the group consisting of Zn and Ga, and a support material, wherein the two metals are present in an intermetallic composition, wherein the group A metal is Ni, and the group B metal is Ga.

The metal (A) and the metal (B) together form an intermetallic composition deposited on the support material.

The term “intermetallic composition” is defined herein as compositions where two metals in oxidation state (0) are present together in the unit cell of the catalytically active material.

The inventive catalyst has a surprisingly good selective catalytic effect on the hydrogenation of acetylene to ethylene without hydrogenation of the ethylene to ethane. Furthermore the metals from the groups A and B are available to a substantially lower price when compared with the prior art catalysts based on palladium and silver or palladium and gallium.

The present invention further relates to a catalytic process for selectively hydrogenation of acetylene to ethylene of a gas mixture comprising one or more of acetylene, ethylene, carbon monoxide and a stoichiometric excess of hydrogen with use of the inventive catalyst.

In one aspect, the invention provides an intermetallic catalyst AB consisting of the metal A which may be Fe, Co or Ni, and a metal B which may be Zn or Ga on a support C having a regular lattice structure and selected from the group of oxides, typically MgAl₂O₄, Al₂O₃, SiO₂, ZrO₂, TiO₂, CaCO₃ and others, wherein the group A metal is Ni, and the group B metal is Ga. The resulting bimetallic catalyst is capable of selectively hydrogenating unsaturated hydrocarbons using H₂ as effectively as conventional bimetallic Pd-based catalyst.

In another aspect, the invention provides an intermetallic catalyst AB consisting of the metal A which may be Fe, Co or Ni, and a metal B which may be Zn or Ga on a support C having a regular lattice structure and selected from the group of oxides, typically MgAl₂O₄, Al₂O₃, SiO₂, ZrO₂, TiO₂, CaCO₃ and others, wherein the group A metal is Ni, and the group B metal is Ga, wherein the total amount of the group A metal an the group B metal is between 0.1 and 30 wt. % calculated as the elemental metals based on the total mass of the catalyst. The resulting bimetallic catalyst is capable of selectively hydrogenating unsaturated hydrocarbons using H₂ as effectively as conventional bi-metallic Pd-based catalyst.

The present invention further relates to a method for the manufacture of a catalyst which comprises the steps of:

(1) preparing a solid composition by
contacting a liquid composition (a) comprising a metal A selected from the group of Fe, Co and Ni with a liquid composition (b) comprising a metal B selected from the group of Zn and Ga, wherein the group A metal is Ni, and the group B metal is Ga and (c) an inorganic support comprising Al₂O₃, SiO₂, ZrO₂, TiO₂, CaCO₃, MgAl₂O₄ or mixtures thereof, or
contacting a single liquid composition comprising a metal A selected from the group of Fe, Co and Ni and a metal B selected from the group of Zn and Ga, wherein the group A metal is Ni, and the group B metal is Ga, with (c) an inorganic support comprising Al₂O₃, SiO₂, ZrO₂, TiO₂, CaCO₃, MgAl₂O₄ or mixtures thereof; and
   (2) drying, calcining, and optionally reducing the contacted solid composition obtained by step (1).

The term “calcining” is defined herein to be a thermal treatment process applied to metal compounds in order to bring about a thermal decomposition or removal of volatile fractions. The calcination process normally takes place at temperatures below the melting point of the product materials.

The impregnating solution may comprise both the metal compound or precursor A and a metal compound or precursor B, such that the metals are preferably applied to the support together and at the same time. In this embodiment, the drying, calcining, and reducing steps may preferably be conducted once.

The present invention further relates to a use of a catalyst according to the invention for the selective hydrogenation of acetylenic impurities (preferably present in a small amount in an ethylene-containing gas stream) with hydrogen gas carried out with a catalyst prepared by the methods described herein.

Furthermore the present invention relates to a method of catalytic selective hydrogenation of acetylene to ethylene in a gas mixture wherein the catalytic hydrogenation is carried out with use of the inventive catalyst.

### DETAILED DESCRIPTION OF THE INVENTION

The present invention concern an alternative catalyst for selective hydrogenation of acetylene to ethylene prepared from substantially cheaper materials as compared with the prior art catalysts with similar catalytic activity based on expensive platinum group metals such as Pd.

Thus, the present invention provides a catalyst for selective hydrogenation of acetylene to ethylene including a metal (A), which may be iron, cobalt or nickel and a metal (B), which may be zinc or gallium, where the metals are deposited on a catalyst support (C), wherein the group A metal is Ni, and the group B metal is Ga.

It is preferred that the total amount of the group A metal and the group B metal is between 0.1 and 30 wt. %, more preferred 5-20 wt. %, and most preferred 7-18 wt. %, calculated as the elemental metals based on the total mass of the catalyst, i.e. the total mass of the elemental metal A+the elemental metal B+support. The resulting bimetallic catalyst is cheap and capable of effectively and selectively hydrogenating acetylene using a reducing gas such as H₂.

It is also preferred that the catalyst composition has a metal A content of about 8 to about 35 wt. % based on the total amount of the metal A and the metal B calculated as the elemental metals. The resulting bimetallic catalyst is cheap capable of effectively and selectively hydrogenating acetylene using a reducing gas such as H₂.

The supported intermetallic catalyst AB according to the invention includes intermetallic compositions of Ni+Ga.

The support or support material (C) according to the invention may be selected from the group of oxides, typically MgAl₂O₄, Al₂O₃, SiO₂, ZrO₂, TiO₂, CaCO₃ and others or mixtures thereof.

In yet another aspect of the invention relates to a preparation method comprising the steps of: (1) contacting a liquid composition (a) comprising a metal A selected from the group consisting of Fe, Co or Ni with a liquid composition (b) comprising a metal B selected from the group consisting of Zn or Ga or a single liquid composition (a+b) comprising a metal A selected from the group consisting of Fe, Co or Ni and a metal B selected from the group consisting of Zn or Ga, wherein the group A metal is Ni, and the group B metal is Ga, and (c) an inorganic support comprising Al₂O₃, SiO₂, ZrO₂, TiO₂, CaCO₃, MgAl₂O₄ or mixtures thereof, so as to produce a contacted solid composition; and (2) drying, calcining, and optionally reducing the contacted solid composition obtained by step (1).

In another preferred embodiment, the impregnating solution may comprise both the metal compound or precursor A and the metal compound or precursor B, such that the metals are preferably applied to the support C together and at the same time. In this embodiment, the drying, calcining, and reducing steps may preferably be conducted once.

Another preferred embodiment of the present invention includes a catalyst for selective hydrogenation wherein a supported catalyst comprising metal A or metal B is obtained commercially and further prepared as described herein preferably by wet impregnation with a promoter metal or metal precursor, although the promoter metal may be applied by any technique known in the art without departing from the scope of the invention.

Satifactory results are achieved with such metal salts as, without limitations, the soluble nitrates and such organic salts as oxalates, acetates and formates. The amount of metal compound (A or B) in the impregnating solution is a ready means of controlling the quantity of metal deposited on the support and the upper limit of metal compound deposited is determined by the solubility of the metal compounds used.

The preferred duration of the impregnating step is that which is necessary to produce the desired equilibrium of distribution of the metal between the impregnating solution and the solid support. As will be understood, within reasonable limits, it is possible to use an impregnating solution somewhat more concentrated than that theoretically necessary in order to reduce the time for complete impregnation and without imparing the selectivity of the catalyst material produced. In most instances however, the satifactory time for impregnation varies generally from about 8-24 hours.

The reducing gas is preferably hydrogen or a hydrogen-containing gas, as will be known to those skilled in the art, and may contain carbon monoxide or a carbon monoxide-containing gas. Both the drying and calcining steps may take place in oxygen-containing or substantially oxygen-free environments.

The catalyst support (C) is preferably MgAl₂O₄, Al₂O₃, SiO₂, ZrO₂, TiO₂, CaCO₃ or mixtures thereof. The most preferred support is MgAl₂O₄ (spinel).

The catalyst particles can be prepared by any suitable means. The metals (A and B) may be added to the support (C) by any means known in the art, such as, without limitations, impregnation, incipient wetness impregnation, deposition, chemical-vapor deposition, immersion and spraying. The metal salts utilized herein can be substituted by any of the well-known water soluble salts or complexes that decompose to oxides when heated in air. The sequence of the impregnation of support by metal components to obtain bimetallic catalysts may also be altered in a wide range.

Thus, the components A and B may be deposited onto and/or incorporated into the inorganic support material (C) by any suitable means.

In another aspect, the present invention further includes the use/application of the catalysts as described herein to selective conversion of acetylenic compounds to ethylenic compounds comprising the charging of a feed-stream containing the acetylenic compound or compounds to a single pass, continuous reactor containing the catalyst of this invention and operated at conditions conductive to hydrogenation. The acetylenic compound may be a gas and the reactor may be operated such that the fluid media in the reactor is in the gas or supercritical fluid phase form. The acetylenic compound may alternatively be a liquid and distributed as a component of a stream wholly or mostly in the gas state at reactor operating conditions such that the fluid media in the reactor is in a gas, supercritical, or mixed phase form. Further, alternatively, the acetylenic compound may be a liquid and distributed as a component of a stream wholly or mostly in the liquid state at reactor operating conditions such that the fluid media in the reactor is in the liquid, supercritical, or mixed phase form. Also, the acetylenic compound may be a gas at reactor operating conditions and distributed as a component of a stream wholly or mostly in the liquid state such that the fluid media in the reactor is in a liquid, supercritical, or mixed phase form.

In preferred embodiment, the present invention further includes the use/application of the catalysts as described herein for the selective hydrogenation of acetylene to ethylene in a gas mixture.

In yet another preferred embodiment, the present invention further includes the use/application of the catalysts as described herein for the selective hydrogenation of acetylene to ethylene in a gas mixture, wherein said gas mixture comprises acetylenic impurities and hydrogen and one or more of, ethylene and carbon monoxide.

In another preferred embodiment, the selective acetylene hydrogenation process of this invention is carried out by contacting a) feed gas which comprises acetylene, preferably an ethylene stream containing acetylene as an impurity and b) hydrogen gas with c) the catalyst composition(s) of the present invention. Gases a) and b) may be premixed before their contact with the catalyst composition c). It is within the scope of this invention to have additional gases, including, for example, as ethane, carbon monoxide, present in the feed gas, as long as they do not significantly interfere with the selective hydrogenation of acetylene to ethylene. Generally, carbon monoxide and ethane are present in trace amounts (typically less than 1 vol % and 5 vol percent, respectively). The temperature at which the selective hydrogenation of acetylene to ethylene is carried out in the invention depends Largely on the activity of the catalysts and the extent of acetylene removal desired. Generally temperatures in the range between about 30 to about 550° C.

The carrier or support material C on which the active catalyst metals from group A and group B are supported is preferably an oxide carrier, said carrier being formed to have a surface area greater than 10 m²/g, preferably greater than 50 m²/g, more preferred above 100 m²/g.

Preferably the oxide carrier is selected from the group consisting of MgAl₂O₄, Al₂O₃, SiO₂, ZrO₂, TiO₂, CaCO₃ and mixtures thereof. The most preferred support is Al₂O₃ and MgAl₂O₄ (spinel).

The amount of the metal or metals from group A is between 0.1 and 60 wt. %, preferably 3-50 wt. %, more preferred 5-40 wt. %, most preferred 8-35 wt. %, based on the total amount of the metals form group A and group B calculated as the elemental metals.

The total amount of the metal or metals from group A and the metal or metals from group B is between 0.1 and 30 wt. %, preferably 5-20 wt %, most preferred 7-18 wt %, calculated as the elemental metals and based on the total weight of the catalyst. As will be understood by a person skilled in the art an interval of 0.1 to 30 wt. %, as mentioned above, comprise without limitation 1.0 to 28 wt. %, 2.5 to 25 wt. %, 4 to 23 wt. %, 3 to 17 wt. %, 5 to 15 wt. % and 7 to 13 wt. % as well as other intervals falling within this range.

In a preferred embodiment the hydrogenation by the inventive process can be carried out at a pressure in the range from 0.1 to 10 MPa (1 to 100 bar), preferably above 0.5 MPa (5 bar) such as 0.5-10 MPa, 1-5 MPa, or 2-4 MPa (5-100 bar, 10-50 bar, or 20-40 bar), and at a temperature between 30° C. and 550° C.

The gas mixture to be treated by the inventive process typically has a concentration of hydrogen of 1.1-4 vol parts, preferably 1.3-3 vol parts more preferred 1.5-2 vol parts, per one vol part of acetylenic impurities.

The inventive catalyst is obtainable by a per se conventional process by impregnating the selected metals from groups A and B on the carrier or a precursor material and heating to above 350° C. in air. Typically, the calcination temperature is 375-525° C., preferably 400-500° C.

The present invention will be described herein through exemplary embodiments and examples. Any embodiments, examples and related data presented here are given just to exemplify the principles of the invention, and are not intended to limit the scope of the invention.

The catalyst may be used in exemplary process for removing or substantially reducing the quantity of unsaturated hydrocarbons in a mixture of gases containing hydrogen, acetylenic impurities, ethylene and carbon monoxide. The process involves passing a mixture of gases over the catalyst at a temperature typically above 25° C., such as 25° C.-550° C., 50° C.-500° C., or 100° C.-400° C.

The inventive catalyst is primarily developed for selective hydrogenation of acetylene to ethene. However any acetylenic impurity can be hydrogenated to the corresponding alkenes as well.

The selective catalytic hydrogenation may be carried out with a reducing gas such as hydrogen, for example using a hydrogen containing gas such as formier gas that is a mixture of up to 10% hydrogen in nitrogen. Other hydrogen containing or hydrogen donating materials (reducing gases) usable for catalytic hydrogenation are also contemplated.

Non-limiting examples of hydrogen donating materials are ammonium formate (NH₄HCO₂) or formic acid (HCOOH).

The term “acetylenic impurities” as used in the present specification and claims includes in principle any alkyne, and in particular acetylene (=ethyne) and in some extent also lower alkynes such as propyne, butyne and pentyne.

The following examples illustrate and explain the details of the present invention, but should not be taken as limiting the present invention in any regard.

### EXAMPLES

### Catalyst Preparation

A number of experimental catalysts were prepared by incipient wetness impregnation, the impregnating solution comprising both the metal compound or precursor A and the metal compound or precursor B, such that the metals are preferably applied to the support C together and at the same time, after which they are dried, calcined, and reduced.

### Example 1

Catalysts containing Ni or Ni and Zn were prepared to have approximately 10 wt. % metal on a 250-500 mesh MgAl₂O₄ spinel carrier. For these catalysts, the carrier was incipient wetness impregnated with a solution containing in total 0.0018 mole/g spinel of Zn(NO₃)₂*6H₂O (Fluka≧99.0%) and Ni(NO₃)₂*6H₂O (Aldrich 99.999%). The impregnated samples were dried at 110° C. before calcination in static air at 450° C. for 4 hours. Catalysts containing Pd or Pd and Ag were prepared in the same way, but with a total of 0.000095 mole/g spinel of Pd(NO₃)₂*2H₂O (Fluka, purum) and AgNO₃ (63% Ag). This corresponds to app. 1 wt. % metal in the final catalysts.

### Catalyst Selective Hydrogenation Screening Tests

Before catalytic testing, 100 mg of the calcined catalyst was placed in a tubular quartz reactor and reduced in a flow of Formier gas (50 mL/min) at 500° C. for 5 hours. After cooling to room temperature, a flow of 300 ml/min reactant gas was applied over the catalyst. The reactant gas stream contained 1.33% C₂H₄, 0.0667% C₂H₂ and 0.67% H₂ in Ar and N₂, which was mixed (using calibrated Brooks Mass Flow Controllers) from Ar (Strandmøllen), 10.008% ethylene and 0.499% acetylene in Ar (Air Products, certified gas mixture) and Formier gas (10% H₂ in N₂, Strandmøllen). For catalytic testing, the catalyst temperature was varied, and gas compositions were measured before and after reaction by GC (Shimadzu GC-17A with a FID detector). GC peak areas were calibrated for ethylene, acetylene and ethane using gasses mixed by Brooks Mass Flow Controllers. These calibrations were used for converting measured peak areas to gas phase concentrations.

### Example 2

### Comparative

Catalyst containing 1 wt. % of metal (100% Pd) on MgAl₂O₄. A catalyst was prepared according to Example 1, that contained 1 wt. % of metal (100% Pd) on MgAl₂O₄ was used for this Example.

100% Pd

| % Acetylene removed, | % Ethane |
|---|---|
| compared to initial amount of acetylene | formed |
| 21.8 | 0.0 |
| 24.8 | 0.0 |
| 29.4 | 0.0 |
| 39.8 | 0.0 |
| 44.9 | 0.0 |
| 46.3 | 0.0 |
| 47.2 | 0.0 |
| 49.1 | 0.0 |
| 52.1 | 0.0 |
| 59.5 | 0.0 |
| 62.0 | 0.0 |
| 77.3 | 0.0 |
| 80.6 | 0.1 |
| 99.6 | 0.4 |

### Example 3

### Comparative

Catalyst containing 1 wt. % of metal (25% Pd and 75% Ag) on a MgAl₂O₄ support. A catalyst was prepared according to Example 1, that contained 1 wt. % of metal (25% Pd and 75% Ag) on MgAl₂O₄ was used for this Example.

25% Pd, 75% Ag

| % Acetylene removed, | % Ethane |
|---|---|
| compared to initial amount of acetylene | formed |
| 12.6 | 0.0 |
| 13.4 | 0.0 |
| 15.8 | 0.0 |
| 18.5 | 0.0 |
| 28.1 | 0.0 |
| 40.1 | 0.0 |
| 53.7 | 0.0 |
| 68.7 | 0.0 |
| 82.3 | 0.0 |
| 90.1 | 0.0 |
| 94.8 | 0.0 |
| 96.8 | 0.0 |
| 97.8 | 0.0 |
| 98.1 | 0.0 |
| 98.7 | 0.0 |

### Example 4

Catalyst containing 10 wt. % of metal (45% Ni and 55% Zn) on a MgAl₂O₄ support. A catalyst was prepared according to Example 1, that contained 10 wt. % of metal (45% Ni and 55% Zn) on MgAl₂O₄ was used for this Example.

45% Ni and 55% Zn

| % Acetylene removed, | % Ethane |
|---|---|
| compared to initial amount of acetylene | formed |
| 2.8 | 0.0 |
| 8.9 | 0.0 |
| 10.0 | 0.0 |
| 13.1 | 0.0 |
| 20.5 | 0.0 |
| 36.4 | 0.0 |
| 43.1 | 0.0 |
| 59.4 | 0.0 |
| 66.9 | 0.0 |
| 79.0 | 0.0 |
| 88.2 | 0.1 |
| 91.2 | 0.2 |
| 93.2 | 0.3 |

### Example 5

Catalyst containing 10 wt. % of metal (33% Ni and 66% Zn) on a MgAl₂O₄ support. A catalyst was prepared according to Example 1, that contained 10 wt. % of metal (33% Ni and 66% Zn) on MgAl₂O₄ was used for this Example.

33% Ni and 66% Zn

| % Acetylene removed, compared | % Ethane |
|---|---|
| to initial amount of acetylene | formed |
| 5.6 | 0.0 |
| 24.1 | 0.0 |
| 63.7 | 0.0 |
| 71.2 | 0.0 |
| 74.7 | 0.0 |
| 83.5 | 0.0 |
| 85.7 | 0.1 |
| 88.0 | 0.1 |
| 90.2 | 0.1 |
| 92.8 | 0.1 |
| 94.8 | 0.1 |
| 96.0 | 0.1 |
| 96.0 | 0.1 |
| 96.4 | 0.1 |
| 99.8 | 0.2 |

### Example 6

Catalyst containing 10 wt. % of metal (25% Ni and 75% Zn) on a MgAl₂O₄ support. A catalyst was prepared according to Example 1, that contained 10 wt. % of metal (25% Ni and 75% Zn) on MgAl₂O₄ was used for this Example.

25% Ni and 75% Zn

| % Acetylene removed, | % Ethane |
|---|---|
| compared to initial amount of acetylene | formed |
| 9.3 | 0.0 |
| 17.0 | 0.0 |
| 14.6 | 0.0 |
| 16.1 | 0.0 |
| 21.9 | 0.0 |
| 35.6 | 0.0 |
| 38.6 | 0.0 |
| 48.1 | 0.0 |
| 61.9 | 0.0 |
| 78.9 | 0.0 |
| 87.8 | 0.0 |
| 91.6 | 0.0 |
| 94.9 | 0.0 |
| 96.2 | 0.0 |
| 97.6 | 0.0 |

The above description of the invention reveals that it is obvious that it can be varied in many ways. Such variations are not to be considered a deviation from the scope of the invention, and all such modifications which are obvious to persons skilled in the art are also to be considered comprised by the scope of the succeeding claims.

1-20. (canceled)

21. A catalyst comprising a mixture of a Ni and Ga, and a support material, wherein Ni and Ga are present in the catalyst as one or more intermetallic compositions.

22. The catalyst according to claim 21, wherein the total amount of Ni and Ga is between 0.1 and 30 wt. % calculated as the elemental metals based on the total mass of the catalyst.

23. The catalyst according to claim 21, wherein the total amount of Ni and Ga is between 5 and 20 wt. % calculated as the elemental metals based on the total mass of the catalyst.

24. The catalyst according to claim 21, wherein the total amount of Ni and Ga is between 7 and 18 wt. % calculated as the elemental metals based on the total mass of the catalyst.

25. The catalyst according to claim 21, wherein the support material is selected from the group consisting of Al₂O₃, SiO₂, ZrO₂, TiO₂, CaCO₃, MgAl₂O₄, and mixtures thereof.

26. The catalyst according to claim 21, wherein the catalyst composition has a Ni content of 8 to 35 wt. % based on the total amount of Ni and Ga calculated as elemental metals.

27. A method for the manufacture of a catalyst, comprising:
(1) preparing a solid composition, comprising
contacting a single liquid composition comprising Ni and Ga, with an inorganic support comprising Al₂O₃, SiO₂, ZrO₂, TiO₂, CaCO₃, MgAl₂O₄, or mixtures thereof; and
(2) drying, calcining, and reducing the solid composition obtained by step (1).

28. A process of catalytic selective hydrogenation of acetylene to ethylene in a gas mixture wherein the catalytic hydrogenation is carried out with use of a catalyst according to claim 21.

29. The process according to claim 28, wherein the gas mixture comprises acetylenic impurities and hydrogen, and one or more of ethylene and carbon monoxide.
